(19) United States
(12) Patent Application Publication
(10) Pub. No.: US 2007/0167395 A1
(43) Pub. Date: Jul. 19, 2007

(54) Compositions and methods for treating diabetes

(76) Inventor: Isaac Eliaz, Sebastopol, CA (US)

Correspondence Address: Steven B. Kelber, Esq., 1875 Eye Street, NW, 5th Floor, Suite 559, Washington, DC, 20005, US

(21) Appl. No.: 11/332,237

(22) Filed: Jan. 17, 2006

(51) Int. Cl. A61K 31/732; A61K 31/729

(52) U.S. Cl. 514/54

(57) ABSTRACT

Low molecular weight modified alginate and/or low molecular weight modified pectin, particularly modified citrus pectin (MCP), is useful in a composition for the treatment of diabetes, when administered to a diabetic individual. For instance, a composition comprising MCP is administered to an individual in an amount sufficient to reduce a parameter associated with diabetes severity, particularly levels of total blood glucose or triglyceride. The composition also may comprise well known pharmacologically acceptable agents, such as sulfured amino acids, cilantro, garlic, minerals, and herbs.

### BACKGROUND

1. Technical Field

This application pertains generally to methods of using compositions comprising modified pectin or modified alginate to treat diabetes.

2. Background of the Technology

Current dietary guidelines of the American Diabetes Association (ADA) emphasize the importance of controlling diabetes in part through consuming starchy staples higher in fiber and having a lower glycemic index. Diets high in cereal fiber have been associated with improved glycemic control. The beneficial effect of fiber is not readily explained but may result from a slower rate of carbohydrate absorption. See Jenkins et al., “High-complex carbohydrate or lente carbohydrate foods?” Am. J Med. 113 (Suppl. 9B): 30S-37S (December 2002). In one study, however, administration of pectin in palatable form delayed gastric emptying of the last 20% of an ingested meal but provided no significant difference in blood glucose levels from a placebo. See Iftikhar et al., “The effect of pectin on the gastric emptying rates and blood glucose levels after a test meal,” J. Pharm. Pharmacol. 46: 851-53 (1994). The recent realization that fiber can be absorbed into the blood stream suggests that fiber may have possible beneficial effects beyond slowing the rate of carbohydrate absorption.

Despite the ADA dietary guidelines, diabetes is becoming more common in the United States: since 1980, the number of Americans with diabetes has more than doubled to over 14 million. People aged 65 years or older account for almost 40% of the population with diabetes. See Center for Disease Control, Prevalence of Diabetes (October 2005), http://www.cdc.gov/diabetes/statistics/prev/national/figpersons.htm. Accordingly, there is an ongoing need for new and more effective treatments for diabetes.

### SUMMARY

The need for new treatments for diabetes is met by the present method of administering an individual suffering from diabetes a composition comprising pectin modified to be in a low molecular weight form, particularly modified citrus pectin (MCP). According to a first embodiment, a method of treating diabetes comprises administering to an individual having diabetes a composition comprising a modified pectin or a modified alginate, where the modified pectin or modified alginate is sufficiently low in molecular weight to be adsorbed into the blood stream following ingestion and is administered in an amount sufficient to treat diabetes. The administration of other pectins, such as pectin oligosaccharide (POS), or low molecular weight modified alginate likewise will be useful in the treatment of diabetes. In one embodiment, the modified pectin or modified alginate has an average molecular weight of 40,000 daltons or less.

In one embodiment of the invention, a composition comprising modified pectin or modified alginate is administered to an individual, which may be a human or another animal. The composition may comprise a modified pectin, such as MCP or POS, alone or combined with a modified alginate, or it may comprise a modified alginate without a modified pectin. The composition preferably comprises modified pectin, and the modified pectin preferably is MCP. The composition may be administered orally or intravenously, or by any other manner effective to provide modified pectin in an amount sufficient to change positively a parameter associated with diabetes severity. In one embodiment, the parameter associated with diabetes is the level of blood glucose, HBAlC, LDL, VLDL, total cholesterol or total triglyceride. Modified pectin and/or modified alginate may be combined with well known pharmacologically acceptable agents, including nutrients, dietary supplements, excipients and potentiating agents, such as vitamins, sulfured amino acids, cilantro, garlic, minerals, and herbs.

According to a second embodiment, the invention provides a method of reducing the risk that an individual will develop diabetes, comprising administering to an individual at risk for developing diabetes a composition comprising a modified pectin or a modified alginate, where the modified pectin or modified alginate is sufficiently low in molecular weight to be adsorbed into the blood stream following ingestion, and where the modified pectin or modified alginate is administered in an amount sufficient to reduce the risk that the individual will develop diabetes. In one embodiment, the modified pectin or a modified alginate has an average molecular weight of 40,000 daltons or less. The reduction of risk of developing diabetes in this embodiment is determined by assessing standard diagnostic measures for the risk of developing diabetes, such as reduced ability to control blood glucose levels. A positive change in any diagnostic indicia of diabetes reflects a reduction of risk in developing diabetes.

### DETAILED DESCRIPTION OF THE PREFERRED EMBODIMENTS

The present invention provides methods for using low molecular weight modified pectin, including MCP and POS, and low molecular weight modified alginate compositions to treat diabetes. While the present invention is not limited by a particular theory of a mechanism of action, it is believed that the presently disclosed therapeutic effect of low molecular weight modified pectins and low molecular weight modified alginates generally is related to their ability to bind various detrimental agents in the circulatory system of the treated individual.

The methods of the present invention provide a composition administered in an amount sufficient to treat diabetes, where the treated individual is already diagnosed with diabetes. The treatment of diabetes in this case may be reflected by a positive change in a parameter associated with diabetes severity, such as levels of blood glucose, HBAlC, total triglycerides, LDL, VLDL, and total cholesterol. For example, treatment according to the present methods may be reflected by a decrease in the level of total blood triglycerides or blood gluocose. The methods of the present invention also may be applied to individuals at risk for developing diabetes, as diagnosed by any standard medical procedure, e.g., an increased inability to control blood glucose levels. In this case, treatment is reflected by a positive change in any diagnostic indicia of diabetes that reflects a reduction of risk in developing diabetes, e.g., an increased ability to control blood glucose.

Methods of manufacturing low molecular weight modified pectins, including MCP and POS, and low molecular weight modified alginates are known in the art and include, for example, U.S. Pat. Nos. 6,274,566 and 6,462,029, incorporated by reference in their entirety herein. These patents disclose, among other things, the manufacture and use of compositions comprising low molecular weight modified pectins, including MCP and POS, and low molecular weight modified alginates for the treatment of a variety of human conditions. These patents particularly disclose the manufacture and use of chemically or enzymatically modified pectins and alginates having an average molecular weight of 40,000 daltons or less.

Algin, pectin, and digestion products of each are known gelling agents. Algins are principally found in seaweed, while suitable pectins can be found in a variety of fruits and vegetables, such as citrus fruit, apples, grapefruit, crabapples, pears, quince, gooseberries, plums, cranberries, beets, mangos and passion fruit. Alginates comprise repeating subunits of polyguluronic acid bound by glycosidic linkages at the 1a-4a-di-axial position and repeating subunits of polymannuronic acid bound by galactic links at the 1e-4e di-equatorial position. Pectins are polymers of galacturonic acid, which may be partially esterified. Naturally occurring algins and pectins are high molecular weight products; however, both can be reduced to low molecular weight products either by chemical treatment, e.g., alkaline hydrolysis, or by enzymatic degradation.

“Modified alginates” and “modified pectins” refer to products obtained by hydrolysis or enzymatic digestion of algin or pectin that are sufficiently low in molecular weight to be adsorbed into the blood stream following ingestion. Modified alginates and pectins in one embodiment have an average molecular weight of 40,000 daltons or less.

Low molecular weight pectins can be obtained from commercial sources, and methods for obtaining low molecular weight pectins are known in the art. See, e.g., Pienta et al., J Nat'l Cancer Inst., 87: 348-52 (1995). Modified alginates are produced by alkaline hydrolysis or enzymatic degradation using alginate lyase, as described in greater detail in U.S. Pat. No. 6,462,029, incorporated herein by reference. The final modified alginate or pectin is water soluble and may have an average molecular weight of 40,000 daltons or less. Preferably, the modified alginate or modified pectin has an average molecular weight between about 10,000 daltons and about 20,000 daltons. More preferably, the modified alginate or modified pectin has an average molecular weight of about 10,000 daltons. The modified alginate or modified pectin also preferably has a degree of esterification of less than about 10%.

Modified alginate or modified pectin may be administered independently or in combination, and they may be combined with a wide variety of pharmaceutically acceptable carriers, conventional excipients, flavorings and the like that are suitable for oral or intravenous administration, depending on the treatment method desired. Modified alginate or modified pectin also may be administered with fruits and vegetables, such as apples, beets and mangos, or in combination with fruit or vegetable juices or purees. Alginates and pectins generally are well tolerated through both methods of administration; neither modified alginate nor modified pectin is known to have any side effects or to exhibit cytopathology or toxicology.

When delivered orally, the modified alginate or modified pectin of the invention is of sufficiently low molecular weight and is sufficiently water-soluble to be readily absorbed through the intestinal mucosa into the bloodstream. Dosage levels will vary from 5-1,500 mg/kg body weight/day and may be sustained over a prolonged period. A preferred range is 10 mg/kg/day to 1,000 mg/kg/day. For example, modified alginate and/or modified pectin powder may be encapsulated into gelatin capsules in the amount of 800 mg/capsule. Alternatively, a water-based preparation may be used, e.g., 6 capsules taken three times a day with 8 full ounces of water or juice. Controlled dosage formulations are preferred to ensure adequate medication over time. Compositions comprising modified pectin and/or modified alginate may be administered over a substantial time period, since the compositions are well tolerated. In one embodiment, the composition may be administered over 4-8 weeks or longer, preferably 6 weeks or more. Oral administration is preferred for such long treatment regimens.

The modified alginate and/or modified pectin may be administered with agents that enhance binding, such as glutathione-rich whey protein, and related binding adjuvants. Additional effective compounds include diuretics or agents that aid excretion through the kidneys or intestines. Other agents that potentiate the effects of the modified alginate and/or modified pectin composition include sulfured amino acids, EDTA, chromium, selenium, vanadium, inducers of phase II detoxification enzymes, cilantro, garlic, minerals, herbs and other botanicals, anti-oxidants, curcumin, indoles, zinc and glutathione. Unmodified (i.e., higher molecular weight, naturally occurring) algin and/or pectin also may be added to the modified alginate and/or modified pectin composition to enhance binding of substances in the intestine.

The following example is intended to be non-limiting and is exemplary of embodiments contemplated but not shown.

### EXAMPLES

1. Background

Animal models of diabetes mellitus are readily available. Diabetic animals are administered with compositions of the present invention, and their blood glucose level and other parameters are monitored. In the following examples, a composition comprising MCP was administered orally to diabetic and control ICR mice at the School of Medicine, Zhejiang University, Hangzhou, China.

1.1. Experimental design:

There were five groups of ICR mice:
Group 1: Normal healthy animals that were administered 0 mg/kg body weight of a composition comprising MCP;
Group 2: Diabetic mice administered 0 mg/kg;
Group 3: Diabetic mice with a daily oral administration of 800 mg/kg;
Group 4: Diabetic mice with a daily oral administration of 1600 mg/kg; and
Group 5: Diabetic mice with a daily oral administration of 2400 mg/kg.

Animals were made diabetic by injecting 100 mg/kg of alloxan in a 2% solution. Blood glucose concentration was measured 72 hours after injection. Animals with blood glucose level between 15 and 30 mmol/L were used for the study. Animals were orally administered MCP for six weeks. Blood glucose concentrations were measured before MCP administration and 6 weeks after administration. At the end of the study, the total cholesterol and total triglyceride levels of the animals also were measured.

1.2. Results:

Table 1 shows the average blood glucose concentrations of the five groups of animals. Group 3 showed a statistically significant difference from the Week 0 and control groups (P<0.01). Group 4 also showed a significant difference (P<0.05), while Group 5 showed no significant difference.

TABLE 1
Animal Blood Glucose Concentration

| | | Blood Glucose | Blood Glucose Conc. |
|---|---|---|---|
| | No. of | Conc. (mmol/L, | (mmol/L, x ± s) at |
| Animal Group | Animals | x ± s) at Week 0 | Week 6 |
| Healthy Control | 30 | 6.68 ± 1.68 | 6.23 ± 1.27 |
| No MCP | | | |
| Diabetes Model | 15 | 22.48 ± 6.38 | 21.45 ± 5.83 |
| No MCP | | | |
| Diabetes Model | 17 | 22.24 ± 5.74 | 14.59 ± 6.55 |
| 800 mg/kg MCP | | | |
| Diabetes Model | 15 | 22.06 ± 5.22 | 16.25 ± 6.31 |
| 1600 mg/kg | | | |
| MCP | | | |
| Diabetes Model | 14 | 22.55 ± 5.63 | 18.52 ± 6.60 |
| 2400 mg/kg | | | |
| MCP | | | |

Table 2 shows the total cholesterol and triglyceride levels of all animals. No significant difference was observed for total cholesterol; however, a significant difference was observed for total triglyceride in Group 3 (P<0.01) and Group 4 (P<0.05), while Group 5 again showed no significant difference.

TABLE 2
Animal Total cholesterol (TC) and Total
Triglyceride (TG) Levels at Week 6

| | No. of | TC Level | TG Level |
|---|---|---|---|
| Animal Group | Animals | (mg/dL, x ± s) | (mg/dL, x ± s) |
| Healthy Control | 30 | 89.86 ± 18.05 | 128.46 ± 46.47 |
| No MCP | | | |
| Diabetes Model | 15 | 100.70 ± 20.07 | 123.31 ± 45.73 |
| No MCP | | | |
| Diabetes Model | 17 | 89.01 ± 14.24 | 77.00 ± 24.51 |
| 800 mg/kg MCP | | | |
| Diabetes Model | 15 | 98.29 ± 27.50 | 92.71 ± 23.91 |
| 1600 mg/kg | | | |
| MCP | | | |
| Diabetes Model | 14 | 91.01 ± 11.31 | 18.52 ± 6.60 |
| 2400 mg/kg | | | |
| MCP | | | |

1.3. Discussion:

This study was designed to see whether administration of a composition comprising MCP reduced blood glucose concentration and a parameter associated with the severity of diabetes. In this case, MCP significantly reduced total triglyceride levels in an animal model of diabetes, indicating that MCP exerted a beneficial effect on a parameter associated with diabetes. A reversed dose response was observed, with the 800 mg/kg group showing the best results. Further studies using the same experimental design can be used to find the optimum dose of MCP required for its beneficial effect.

While the foregoing specification teaches the principles of the present invention with examples provided for the purpose of illustration, the skilled artisan will appreciate from reading this disclosure that various changes in form and detail can be made without departing from the true scope of the invention.

What is claimed is:

1. A method of treating diabetes, comprising administering to an individual having diabetes a composition comprising a modified pectin or a modified alginate, where the modified pectin or modified alginate is administered in an amount sufficient to treat diabetes.

2. The method of claim 1, where the modified pectin or a modified alginate is administered in an amount sufficient to change positively a parameter associated with diabetes severity.

3. The method of claim 1, where the composition comprises modified alginate.

4. The method of claim 1, where the composition comprises modified pectin.

5. The method of claim 4, where the modified pectin is modified citrus pectin (MCP).

6. The method of claim 4, where the modified pectin is pectin oligosaccharide (POS).

7. The method of claim 4, where the composition comprises MCP and POS.

8. The method of claim 2, where the parameter associated with diabetes severity is the level of blood glucose, HBAlC, LDL, VLDL, total cholesterol or total triglycerides in the individual.

9. The method of claim 8, where the composition comprises MCP.

10. The method of claim 1, where the individual is an animal.

11. The method of claim 1, where the individual is a human.

12. The method of claim 1, where the composition is administered orally.

13. The method of claim 1, where the composition is administered intravenously.

14. The method of claim 1, where the composition further comprises a compound selected from the group of agents that enhance binding by modified alginate or modified pectin, whey protein, nutrients, dietary supplements, vitamins, juices or purees of fruits or vegetables, chromium, selenium, vanadium, diuretics, sulfured amino acids, EDTA, inducers of phase II detoxification enzymes, cilantro, garlic, minerals, herbs and other botanicals, anti-oxidants, curcumin, indoles, zinc, glutathione, unmodified algin and unmodified pectin.

15. The method of claim 1, where the modified pectin or modified alginate has an average molecular weight of 40,000 daltons or less.

16. The method of claim 15, where the modified alginate or modified pectin has an average molecular weight between about 10,000 daltons and about 20,000 daltons.

17. The method of claim 16, where the modified alginate or modified pectin has an average molecular weight of about 10,000 daltons.

18. The method of claim 1, where the modified alginate or modified pectin has a degree of esterification of less than about 10%.

19. The method of claim 1, where the modified alginate or modified pectin is administered at an amount of 5-1,500 mg/kg body weight/day.

20. The method of claim 19, where the modified alginate or modified pectin is administered at an amount of 10-1,000 mg/kg body weight/day.

21. The method of claim 1, where the composition is administered to the individual for more than four weeks.

22. A method of reducing the risk that diabetes will develop in an individual, comprising administering to an individual at risk for developing diabetes a composition comprising a modified pectin or a modified alginate, where the modified pectin or modified alginate is administered in an amount sufficient to reduce the risk that the individual will develop diabetes.

23. The method of claim 22, where the modified pectin or modified alginate has an average molecular weight of 40,000 daltons or less.
